(19) United States
(12) Patent Application Publication
(10) Pub. No.: US 2026/0171196 A1
(43) Pub. Date: Jun. 18, 2026

(54) PHYSICAL PROPERTY PREDICTION DEVICE, PHYSICAL PROPERTY PREDICTION METHOD, AND RECORDING MEDIUM

(71) Applicant: NEC Corporation, Minato-ku, Tokyo (JP)

(72) Inventors: Kiichi OBUCHI, Tokyo (JP); Shukichi TANAKA, Tokyo (JP); Kiyohiko TOYAMA, Tokyo (JP); Masahiko ISHIDA, Tokyo (JP); Koichi FUNAYA, Heidelberg (DE); Daniel ONORO RUBIO, Heidelberg (DE)

(73) Assignee: NEC Corporation, Minato-ku, Tokyo (JP)

(21) Appl. No.: 19/114,499

(22) PCT Filed: Sep. 29, 2022
(86) PCT No.: PCT/JP2022/036531

§ 371 Date: Feb. 27, 2026

(51) Int. Cl. G16C 20/30; G16C 20/70; G16C 60/00

(52) U.S. Cl. CPC G16C 20/30; G16C 20/70; G16C 60/00

(57) ABSTRACT

In a physical property prediction device, an input information acquisition means acquires input information indicating a structural formula of a molecule. A three-dimensional information generation means generates three-dimensional information indicating a position, type, bounding of each atom included in the structural formula. A feature generation means configured to generate features of the molecule based on the three-dimensional information. A physical property prediction means predicts physical properties of the molecule using the features generated.

### TECHNICAL FIELD

The present disclosure relates to a technique for generating features of a stereostructure of a molecule.

### BACKGROUND ART

In recent years, with utilization of AI in organic and inorganic chemistry, there has been a growing demand for informatization of molecular structures. Patent Document 1 describes a system that extracts features from the two-dimensional structure of a molecule and outputs characteristic values.

### PRECEDING TECHNICAL REFERENCES

### Patent Document

Patent Document 1: Japanese Laid-open Patent Publication No. 2021-117798

### SUMMARY

### Problem to be Solved by the Invention

In a case of machine learning of molecules with a slightly different molecular structure, as seen in organic chemistry, especially in a polymer field, it is necessary to represent slight differences in a stereostructure to ensure that an amount of information is sufficient and that features are appropriate.

However, for instance, SMILES (Simplified Molecular Input Line Entry System) notations and fingerprints, and other methods until now are based on two-dimensional structure, so a difference in the stereostructure can not be expressed. Therefore, in a case of machine learning in the molecular structure with slightly different stereostructure, it has become a problem that the difference cannot be effectively captured as informative features.

It is one object of the present disclosure to generate features based on the stereostructure of the molecule and select appropriate features.

### Means for Solving the Problem

According to an example aspect of the present disclosure, there is provided a physical property prediction device including:
an input information acquisition means configured to acquire input information indicating a structural formula of a molecule;
a three-dimensional information generation means configured to generate three-dimensional information indicating a position, type, bounding of each atom included in the structural formula;
a feature generation means configured to generate features of the molecule based on the three-dimensional information; and
a physical property prediction means configured to predict physical properties of the molecule using the features generated.

According to another example aspect of the present disclosure, there is provided a physical property prediction method including:
acquiring input information indicating a structural formula of a molecule;
generating three-dimensional information indicating a position, type, bounding of each atom included in the structural formula;
generating features of the molecule based on the three-dimensional information; and
predicting physical properties of the molecule using the features generated.

According to a further example aspect of the present disclosure, there is provided a recording medium storing a program, the program causing a computer to perform a process including:
acquiring input information indicating a structural formula of a molecule;
generating three-dimensional information indicating a position, type, bounding of each atom included in the structural formula;
generating features of the molecule based on the three-dimensional information; and
predicting physical properties of the molecule using the features generated.

### Effect of the Invention

According to the present disclosure, it becomes possible to make slightly different in a stereostructure of a molecule into features, and to appropriately predict a physical property.

### BRIEF DESCRIPTION OF THE DRAWINGS

FIG. 1 illustrates a schematical configuration of a physical prediction system.

FIG. 2 FIG. 2A and FIG. 2B are block diagrams illustrating hardware configurations of a feature generation device and a physical property prediction device.

FIG. 3 illustrates functional configurations of a feature generation device and a physical property prediction device according to a first example embodiment.

FIG. 4A and FIG. 4B illustrate diagrams for explaining variations of monomers in a main chain unit.

FIG. 5 illustrates an example of a data structure of a structural information.

FIG. 6 illustrates an example of divided features of a predetermined molecular group.

FIG. 7 illustrates an example of selection features.

FIG. 8 is a flowchart of a physical property prediction process according to the first example embodiment.

FIG. 9 is a flowchart of a physical property prediction process according to the first example embodiment.

FIG. 10 illustrates a functional configuration of a physical property prediction device according to a second example embodiment.

FIG. 11 is a flowchart of a physical property prediction process according to the second example embodiment.

### EXAMPLE EMBODIMENTS

In the following, example embodiments will be described with reference to the accompanying drawings.

### First Example Embodiment

### Configuration

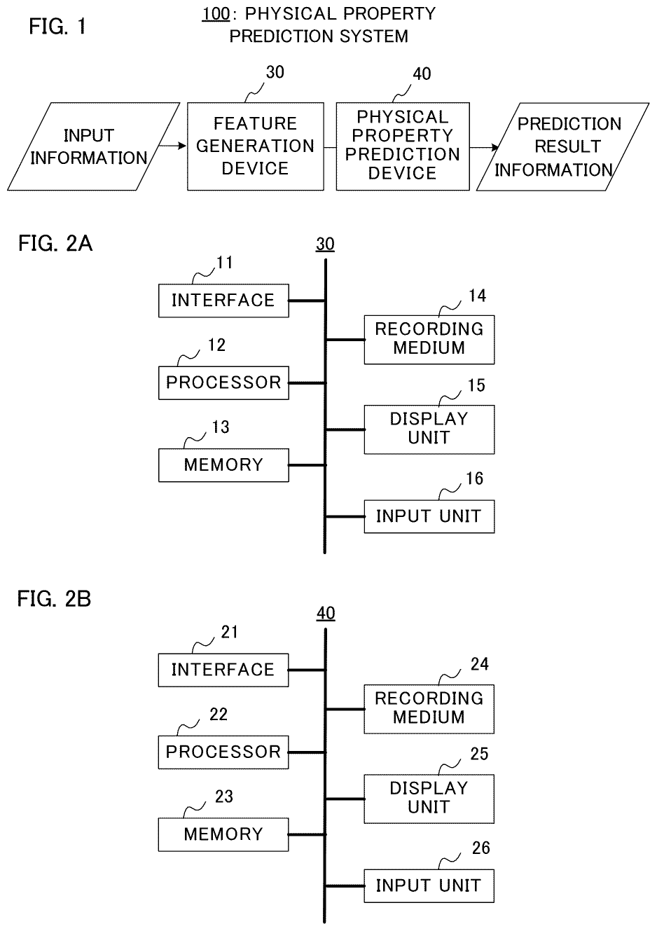

FIG. 1 illustrates a schematic configuration of a physical property prediction system according to a first example embodiment of the present disclosure. A physical property prediction system 100 predicts the physical properties of a molecule based on input information indicating a structural formula of the molecule, outputs prediction result information, and is formed to communicate with a feature generation device 30 and a physical property prediction device 40. Note that in a case where the feature generation device 30 and the physical property prediction device 40 are in separate locations, the feature generation device 30 and the physical property prediction device 40 may be connected via a network such as the Internet. The feature generation device 30 generates structural information (hereinafter, “three-dimensional information”) which indicates a position, type, and bonding of each atom contained in the structural formula of the molecule, and generates and selects features based on the structural information. The physical property prediction device 40 predicts the physical properties of the molecule based on the features, and outputs the prediction result information.

In this example embodiment, the feature generation device 30 and the physical property prediction device 40 are used as separate devices, but the present disclosure is not limited to this configuration and the feature generation device 30 and the physical property prediction device 40 may be formed as a single device. In this case, the single device having functions of the feature generation device 30 and the physical property prediction device 40 is a physical property prediction device 60 of the present disclosure to be described later.

FIG. 2A and FIG. 2B are block diagrams illustrating hardware configurations of the feature generation device 30 and the physical property prediction device 40. First, the feature generation device 30 will be described. As shown in FIG. 2A, the feature generation device 30 includes an interface (Interface) 11, a processor 12, a memory 13, a recording medium 14, a display unit 15, and an input unit 16.

The interface 11 exchanges data with the physical property prediction device 40. The interface 11 is used to transmit the generated and selected features to the physical property prediction device 40. Also, the interface 11 is used when the feature generation device 30 transmits and receives data to and from a predetermined device connected by wire or wireless communications.

The processor 12 is a computer such as a CPU (Central Processing Unit) and controls the entire feature generation device 30 by executing programs prepared in advance. The memory 13 is formed by a ROM (Read Only Memory) and a RAM (Random Access Memory). The memory 13 stores programs executed by the processor 12. Also, the memory 13 is used as a working memory during various processes performed by the processor 12.

The recording medium 14 is a non-volatile and non-transitory recording medium such as a disk-shaped recording medium or a semiconductor memory and is configured to be detachable with respect to the feature generation device 30. The recording medium 14 records various programs to be executed by the processor 12. In a case where the feature generation device 30 executes a physical property prediction process, a corresponding program recorded in the recording medium 14 is loaded into the memory 13 and executed by the processor 12.

The display unit 15 is, for instance, an LCD (Liquid Crystal Display) or the like, and displays a stereochemical formula etc. inputted as input information. The input unit 16 is a keyboard, a mouse, a touch panel, or the like, and is used by a user to input the information etc.

Next, the physical property prediction device 40 will be described. As illustrated in FIG. 2B, the physical property prediction device 40 includes an interface (Interface) 21, a processor 22, a memory 23, a recording medium 24, a display unit 25, and an input unit 26.

The interface 21 exchanges data with the feature generation device 30. The interface 21 is used to receive information of features from the feature generation device 30. Also, the interface 21 is used when the physical property prediction device 40 transmits and receives data to and from a predetermined device that is connected by wire or wireless communications.

The processor 22 is a computer such as a CPU, to control the entire physical property prediction device 40 by executing a predetermined program. The memory 23 is formed by a ROM, a RAM or the like. The memory 23 stores programs executed by the processor 22. The memory 23 is also used as a working memory during various processes performed by the processor 22.

The recording medium 24 is a non-volatile and non-transitory recording medium such as a disk-shaped recording medium or a semiconductor memory and is configured to be detachable with respect to the physical property prediction device 40. The recording medium 24 records various programs executed by the processor 22. In a case where the physical property prediction device 40 executes the physical property prediction process, a corresponding program recorded in the recording medium 24 is loaded into the memory 23 and executed by the processor 22. The display unit 25 is, for instance, an LCD, and displays the prediction result information is a result from predicting the physical properties of the molecule based on the features. The input unit 26 is a keyboard, a mouse, a touch panel, or the like, and is used by the user.

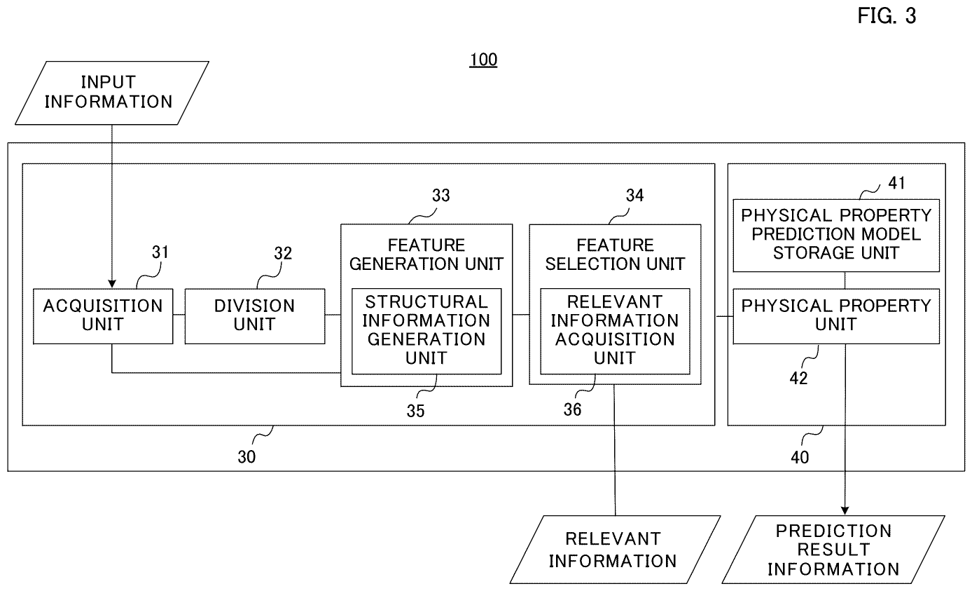

FIG. 3 is a block diagram illustrating functional configurations of the feature generation device 30 and the physical property prediction device 40. The feature generation device 30 generates features based on a stereostructure (hereinafter, also referred to as a “three-dimensional structure”) of a molecule, and selects and extracts appropriate features from the generated features. The feature generation device 30 functionally includes an acquisition unit 31, a division unit 32, a feature generation unit 33, and a feature selection unit 34. The acquisition unit 31, the division unit 32, the feature generation unit 33, and the feature selection unit 34 are realized by the processor 12 executing corresponding programs.

The acquisition unit 31 acquires the input information indicating the structural formula of the molecule. Specifically, the input information indicates a three-dimensional structural formula of a molecule to be a target (hereinafter, also referred to as a “target molecule”) such as a compound name, an image of the structural formula, etc., that is, information capable of expressing the three-dimensional structural formula. In this example embodiment, the input information is input by the user. However, the present disclosure is not limited thereto, and the input information may be acquired from another terminal. In addition, the target molecule may be organic or inorganic, and may be either a compound or a single substance.

A polymer is a molecule with a large molecular weight in a substance and is a compound formed by polymerization of a plurality of monomers. The monomer is a small molecule that serves as a building block of a polymer and is also known as a monomeric unit. Also, a polymer may consist of a main chain and side chains. The main chain is a sequence of molecules that forms a structural backbone of the polymer, while the side chains are molecular chains branching from the main chain.

The division unit 32 divides the target molecule into predetermined units. The target molecule may be divided into a monomer, a dimer, and another oligomeric unit, or may be divided into the main chain unit and the side chain unit, which is divided from the target molecule. Also, division by the division unit 32 includes a function for dividing the target molecule into each synthetic raw material. Although the above are examples of division, but the present disclosure is not limited to these examples.

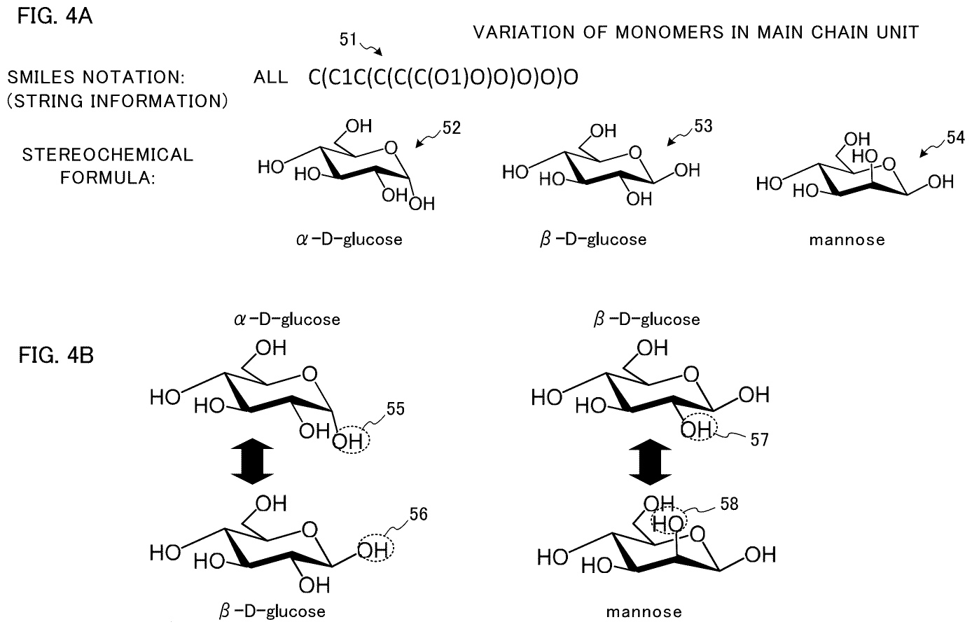

Here, the stereostructure of the monomer of the main chain unit will be described in detail. FIG. 4A and FIG. 4B are diagrams illustrating variations of the monomer of the main chain unit. For instance, as shown in FIG. 4A, α-D-glucose is represented by a string 51 in SMILES notation and is represented by a structural formula 52 in a stereostructure formula. Moreover, β-D-glucose is represented by the string 51 in the SMILES notation and is represented by a structural formula 53 in a stereostructure formula. Furthermore, the mannose (β-D-mannose) is represented by the string 51 in a notation and is represented by a structural formula 54 in the stereostructure formula.

The SMILES notation is the chemical construction of the numerator described by a method of the SMILES notation, which is one of the linear notation methods. Since the SMILES notation is based on the two-dimensional structure of a molecule, the SMILES notation cannot represent differences in stereostructure. As a result, even though α-D-glucose, β-D-glucose, and mannose have different physical properties, they are all represented by the same string 51. However, α-D-glucose and β-D-glucose differ in an orientation of a hydroxy group (—OH) attached to a carbon at one location. Specifically, as shown in FIG. 4B, a hydroxy group 55 of α-D-glucose and a hydroxy group 56 of β-D-glucose have different orientations. The structures of α-D-glucose and β-D-glucose are the same except for the orientation of the hydroxy group 55 and the hydroxy group 56. Similarly, a hydroxyl group 57 of β-D-glucose and a hydroxyl group 58 of mannose have different orientations, while the rest of their structures are the same. Since the input information can represent the stereostructural formula of the target molecule, even minor structural differences can be represented.

The feature generation unit 33 generates features of the target molecule by generating divided features based on each unit divided by the division unit 32 and integrating sets of divided features. The feature generation unit 33 has a structural information generation unit 35. The structural information generation unit 35 generates the structural information indicating the position, type, and bonding of each atom contained in the stereostructural formula which represents a collection of moleculars for each unit (hereinafter, also referred to as a “molecular group”).

Note that for some groups of molecules, the position and bonding of each atom may be adequately represented using two-dimensional information. In that case, the structural information concerning the molecule group is not limited to the three-dimensional information, and may be the two-dimensional information. However, at least one of the molecular groups constituting target molecule S has the structural information that is the three-dimensional information.

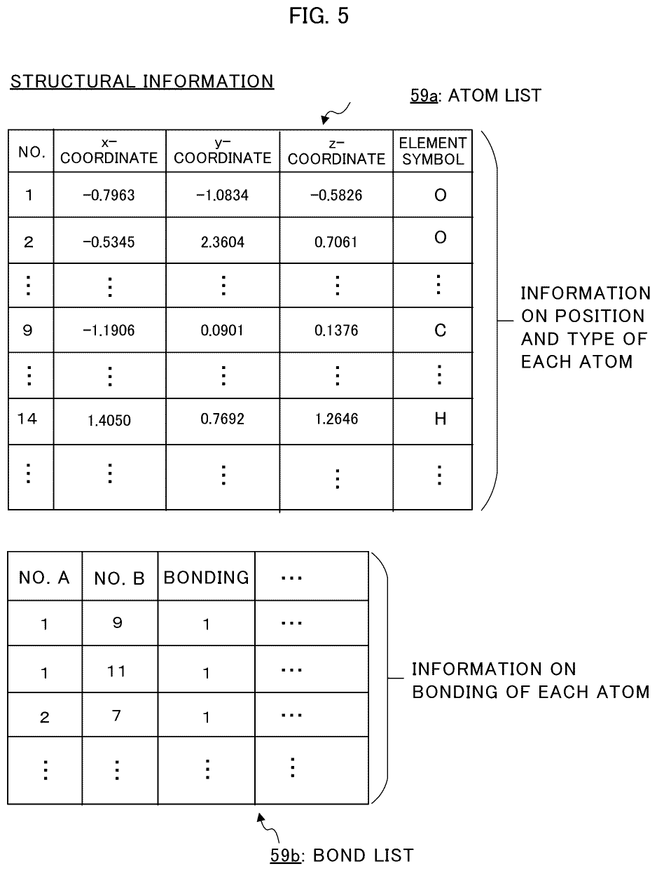

FIG. 5 is an example of a data structure of the structural information. The structural information includes an atom list 59a, which provides information on the position and type of each atom, and a bond list 59b, which provides information on the bonding of each atom. As shown in FIG. 5, the atom list 59a records information on “NO.”, “x-COORDINATE”, “y-COORDINATE”, and “z-COORDINATE”, and “ELEMENT SYMBOL” in association. Here, “NO.” represents a number which identifies each atom constituting a molecular group. Moreover, “x-COORDINATE”, “y-COORDINATE”, and “z-COORDINATE” are information indicating the position of an atom in a given space. Also, “ELEMENT SYMBOL” represents information indicating a type of the atom. Moreover, the bond list 59b stores information of NO. A, NO. B, bonding, etc., as shown in FIG. 5. The “NO.” represents a number which identifies each atom in common with the atom list 59a, and the bond list 59b stores the numbers of bonded atoms and their corresponding types of bonds such as single bonds and double bonds. For instance, a MOL file etc. can be applied to the structural information, but the present disclosure is not limited thereto, and any data can be applied as long as the position, type, and bonding of each atom constituting the molecular group can be represented.

The feature generation unit 33 generates the divided features which quantify characteristics of each molecule group based on the structural information of each molecule group. Specifically, the feature generation unit 33 generates the divided features of each molecular group by converting the structural information of each molecular group into molecular descriptors using, for instance, a library such as RDKit, mordred, etc. Then, the feature generation unit 33 integrates the divided features for all molecular groups to generate the features of the target molecule.

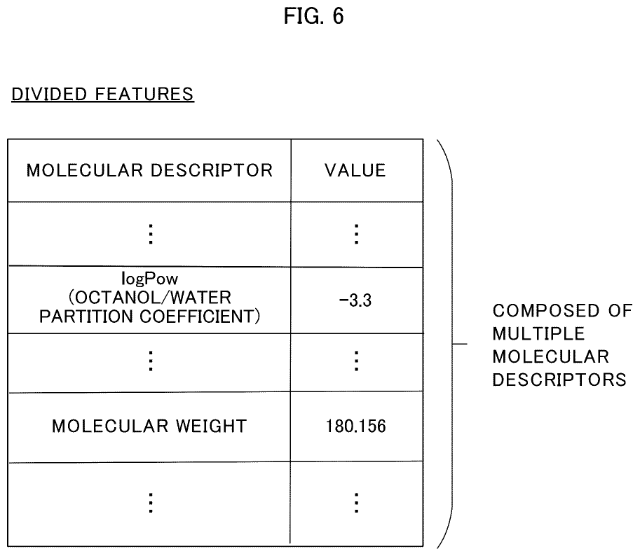

Here, each molecular descriptor is a value which quantifies characteristics of the molecule etc., represents each physicochemical characteristic of each molecule, and includes a value obtained in an experiment or a theoretically calculated value. FIG. 6 illustrates an example of the divided features of a predetermined molecular group. As shown in FIG. 6, the divided features associate respective molecular descriptors such as “octanol/water partition coefficient” obtained experimentally, “molecular weight” calculated theoretically, etc., with their values, and the divided features are formed by a plurality of molecular descriptors.

As described above, by dividing the target molecule into a plurality of units, generating the divided features for each molecular group indicated by each unit, and then integrating all sets of the divided features, it is easy to utilize existing data such as data concerning the main chain and the side chain, and it is possible to efficiently generate the features of the target molecule.

The feature selection unit 34 selects specific features from the features of the target molecule generated by the feature generation unit 33, and extracts the specific features as selection features. The feature selection unit 34 includes a relevant information acquisition unit 36. The relevant information acquisition unit 36 acquires, as relevant information, information related to the target molecule, which is considered in a case of selecting the specific features. In the present example embodiment, relevant information is input by the user, but the present disclosure is not limited thereto and may be acquired from other terminals. Specifically, the relevant information indicates the molecular weight of the target molecule, etc. The feature selection unit 34 selects the selection features by selecting predetermined features while considering the relevant information.

In a case where the feature generation unit 33 converts the structural information into the molecular descriptors using a predetermined library, there are many missing values and duplications in the molecular descriptors which represent the features of the generated target molecule. Here, the missing value means that the molecular descriptor corresponding to the structural information does not exist, a value of the molecular descriptor cannot be calculated, or the like, and the duplication means that the same molecular descriptor and its value exist. Therefore, the feature selection unit 34 extracts, as the selection features, a subset of features from the features of the target molecule that are free of duplications and missing values and that can appropriately represent the three-dimensional structure, including subtle differences, by a predetermined feature selection. The extracted selection features are transmitted to the physical property prediction device 40.

One method of the feature selection is a statistical method using a correlation analysis. For instance, by analyzing a correlation between the features of the target molecule, the feature selection unit 34 identifies features with a degree of correlation equal to or lower than a threshold value as independent features. Then, the feature selection unit 34 selects and extracts the features having the degree of correlation equal to or lower than the threshold value as the selection features which do not duplicate with other features and represents the difference in the stereostructure of the target molecule.

Another method of feature selection is to use a feature selection library such as RandomForest or Boruta. Specifically, the feature selection unit 34 selects and extracts the selection features based on the importance of each feature of the target molecule obtained using the feature selection library. For instance, the feature selection unit 34 may select a number of features corresponding to x % of a sample size used in the feature selection library from the features of the target molecule in an order of importance, and may extract the number of features as the selection features. Moreover, the feature selection unit 34 may select a number of features corresponding to x % of the molecular descriptors which are features of the target molecule, from the features of the target molecule in an order of importance, and may extract the number of features as the selection features. Also, new features may be generated by dimensional compression, such as a principal component analysis.

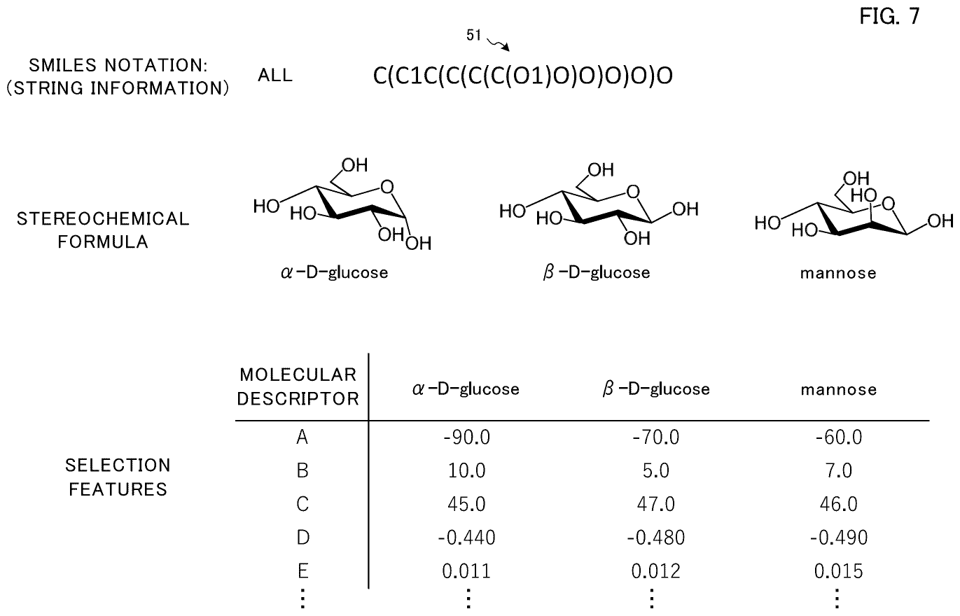

FIG. 7 illustrates examples of the selection features. For instance, although all SMILES notations are described by the same strings 51, the different a-D-glucose, molecular descriptors A to E are different values for α-D-glucose, β-D-glucose, and mannose, which differ in the way the hydroxy groups are attached in their stereostructures, as illustrated in FIG. 7. In this case, the feature selection unit 34 selects and extracts the molecular descriptors A to E and values thereof as the selection features appropriately representing the difference in the stereostructure from the features of the target molecule.

The physical property prediction device 40 predicts the physical properties of the target molecule from the selection features using a physical property prediction model that is trained by machine learning in advance. As illustrated in FIG. 3, the physical property prediction device 40 functionally includes a physical property prediction model storage unit 41, and a physical property prediction unit 42. The physical property prediction model storage unit 41 is realized by the memory 23. The physical property prediction unit 42 is realized by the processor 22 executing a corresponding program.

The physical property prediction model storage unit 41 stores a physical property prediction model which has learned the relationship between the molecular descriptors representing the features of the molecule and physical properties of the molecule. The physical properties are properties of a predetermined molecule. The learning algorithm may use any machine learning technique such as a neural network, SVM (Support Vector Machine), a logistic regression (Logistic Regression), or the like.

Specifically, the physical property prediction device 40 learns the physical property prediction model using training data prepared in advance. Here, the training data include input data and correct data; the molecular descriptors representing the features of the molecule are used as the input data, and correct information concerning the physical properties of the molecule is used as the correct data. The physical property prediction device 40 uses the physical property prediction model to predict the physical properties of the molecule from predetermined input data, and outputs the prediction result information. The physical property prediction device 40 compares the prediction result information output by the physical property prediction model with the correct information concerning the physical properties included in the correct data, to train the physical property prediction model so as to reduce errors.

The physical property prediction unit 42 receives the selection features from the feature generation device 30, and inputs the selection features to the physical property prediction model as the input data, thereby outputting the prediction result information for the physical properties of the target molecule.

### Prediction of Physical Properties

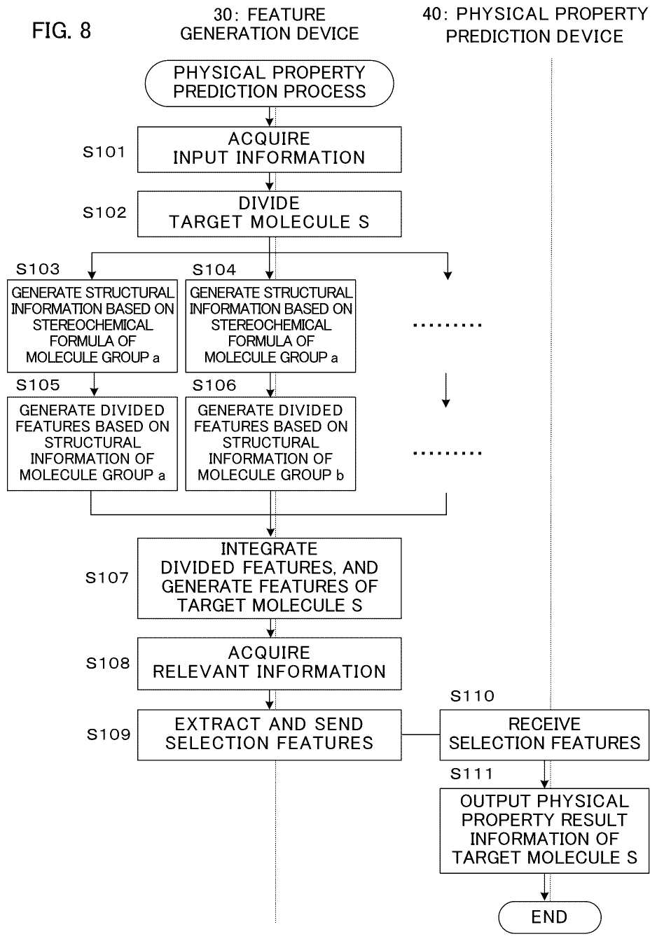

Next, a feature generation process by the physical property prediction system 100 will be described. FIG. 8 is a flowchart of a physical property prediction process performed by the physical property prediction system 100. The feature generation process is realized by the processor 12 of the feature generation device 30 illustrated in FIG. 2A executing a corresponding program prepared in advance. Moreover, the feature generation process is realized by the processor 22 of the physical property prediction device 40 illustrated in FIG. 2B executing a corresponding program prepared in advance.

First, the feature generation device 30 acquires input information indicating the stereochemical formula of the target molecule S upon user input (step S101). Then, the feature generation device 30 divides the target molecule S into a plurality of units such as a molecular group a and a molecular group b based on the input information (step S102). The feature generation device 30 generates the structural information which indicates the position, type, and bonding of each atom contained in the stereochemical formula which represents the molecular group a (step S103). Moreover, the feature generation device 30 generates the structural information which indicates the position, type, and bonding of each atom included in the stereochemical formula which represents the molecular group b (step S104). As described above, the feature generation device 30 generates the structural information of each of the molecular groups from the stereochemical formula indicating each molecular group structure by dividing the target molecule S.

Subsequently, the feature generation device 30 generates the divided features which represent the features quantifying the characteristics of the molecular group a, based on the structural information of the molecular group a (step S105). Specifically, the feature generation device 30 generates the divided features of the molecular group a by converting the structural information of the molecular group a into molecular descriptors by using, for instance, a library such as RDKit or mordred. Moreover, the feature generation device 30 generates the divided features in which the features of the molecular group b are numerically converted based on the structural information of the molecular group b (step S106). Thus, the feature generation device 30 generates the divided features of each molecular group, based on the structural information of each molecular group.

Next, the feature generation device 30 generates features of the target molecule S by integrating all sets of the divided features for all molecular groups (step S107). Moreover, upon user input, the feature generation device 30 acquires the relevant information, such as the molecular weight of the target molecule S, which is considered in a case of extracting the selection features (steps S108). The feature generation device 30 extracts the selection features from the features of the target molecule by a predetermined feature selection based on the relevant information, and transmits the selection features to the physical property prediction device 40 (step S109).

The physical property prediction device 40 receives the selection features from the feature generation device 30 (step S110). Then, the physical property prediction device 40 outputs the prediction result information of the target molecule S by inputting the selection features into the physical property prediction model (step S111). After that, the physical property prediction process is terminated.

As described above, it is possible for the physical property prediction system 100 to extract appropriate features by performing the feature selection after generating a large amount of chemical features based on information of the stereostructure of the molecule. The target molecule can be either organic or inorganic, and can be applied to small molecules, but it is particularly useful in polymer chemistry, especially with polysaccharides.

According to the physical property prediction system 100 of the present disclosure, it is possible to use the difference in the three-dimensional structure of the molecule as the features, and in particular, it is possible to appropriately extract the minor structural differences as the features. Therefore, it is possible to improve the accuracy in machine learning, such as predicting physical properties of materials which contain differences in three-dimensional structure. In other words, the present disclosure can be applied to feature extraction in feature engineering and machine learning, and enables AI utilization for materials development involving differences in stereostructure in organic and inorganic chemistry by using appropriate features.

### First Modification

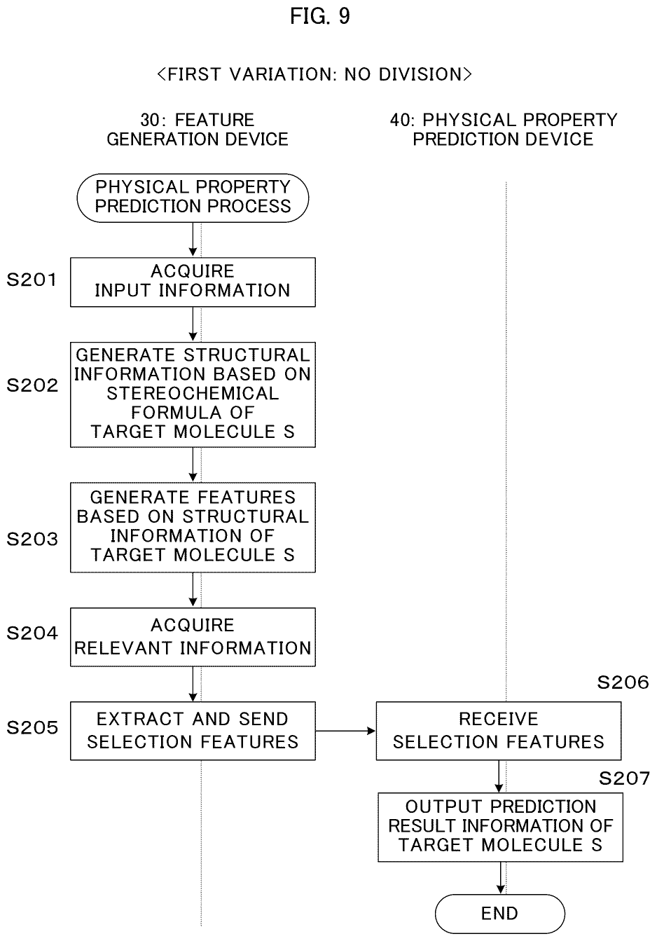

In the first example embodiment described above, the target molecule is divided into a plurality of units by the division unit 32, but the present disclosure is not limited thereto, and the features may be generated without dividing the target molecule. In this case, a feature generation process by the physical property prediction system 100 will be described. FIG. 9 is a flowchart of the physical property prediction process by the physical property prediction system 100 in the first modification. This feature generation process is realized by the processor 12 of the feature generation device 30 illustrated in FIG. 2A executing a corresponding program prepared in advance. Moreover, this feature generation process is realized by the processor 22 of the physical property prediction device 40 illustrated in FIG. 2B executing a corresponding program prepared in advance.

First, the feature generation device 30 acquires the input information which represents the stereochemical formula of the target molecule S upon user input (step S201). Next, the feature generation device 30 generates the structural information which indicates the position, type, and bonding of each atom contained in the stereochemical formula which represents the target molecule S based on the input information (step S202). Subsequently, the feature generation device 30 generates features which quantify the characteristic of the target molecule S based on the structural information of the target molecule S (step S203). Specifically, the feature generation device 30 generates features of the target molecule S by converting the structural information of the target molecule S into molecular descriptors by using, for instance, the library such as RDKit or mordred.

Moreover, the feature generation device 30 acquires the relevant information such as the molecular weight of the target molecule S which is considered in a case of extracting the selection features upon user input (step S204). The feature generation device 30 extracts the selection features from the features of the target molecule by the predetermined feature selection based on the relevant information, and transmits the selection features to the physical property prediction device 40 (step S205).

The physical property prediction device 40 receives the selection features from the feature generation device 30 (step S206). Then, the physical property prediction device 40 outputs the prediction result information of the target molecule S by inputting the selection features into the physical property prediction model (step S207). After that, the physical property prediction process is terminated.

According to this feature generation process, for instance, the present disclosure can be also applied to a low molecular weight monomer or an inorganic compound which does not need to be divided. Moreover, according to this feature generation process, since the feature generation device 30 generates the features without dividing the target molecule S even in a case where the target molecule S is a polymer, it is possible to generate the features reflecting each intermolecular distance and each intermolecular interaction.

### Second Modification

In the first example embodiment described above, a method using the statistical method and the feature selection library is applied as a method of the feature selection, but the present disclosure is not limited thereto, and a method of accepting a selection of features from the user may be applied. In this case, the feature generation device 30 lists the generated features of the target molecule on the display unit 15, and the user selects the selection features from among the features displayed, by the input unit 16. Then, the feature generation device 30 extracts the selection features from the features of the target molecule based on the selection from the user, and performs the physical property prediction process.

At this time, each time a user selects features, the feature generation device 30 may construct a feature selection model by storing selection result information indicating a selection result of the user in association with identification information of the user, and training with training data in which input data indicate the identification information of the user and the correct data indicate the selection result information. According to this manner, in a case where there are sets of the features, it is possible for the feature generation device 30 to select and extract appropriate selection features reflecting a preference of the user by using the feature selection model.

### Second Example Embodiment

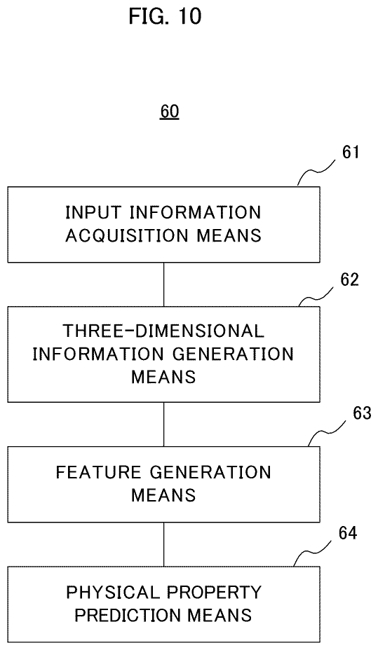

FIG. 10 is a block diagram illustrating a functional configuration of a physical property prediction device of a second example embodiment. The physical property prediction device 60 includes an input information acquisition means 61, a three-dimensional information generation means 62, a feature generation means 63, and a physical property prediction means 64.

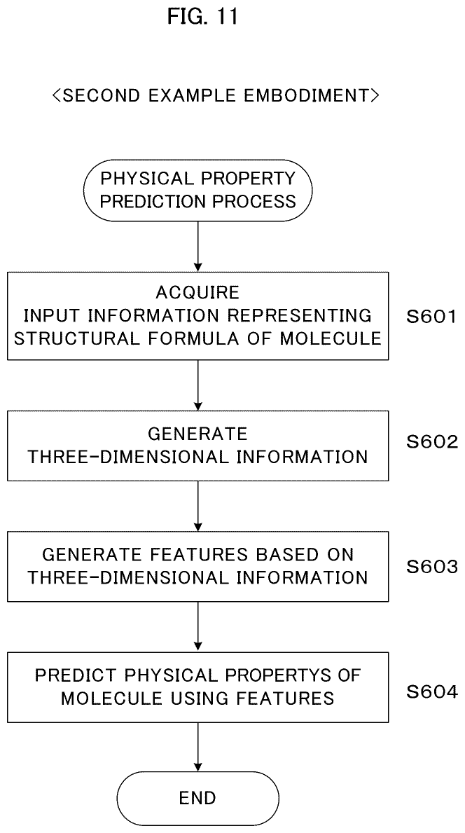

FIG. 11 is a flowchart of a physical property prediction process by the physical property prediction device 60. The input information acquisition means 61 acquires input information indicating the structural formula of the molecule (step S601). The three-dimensional information generation means 62 generates three-dimensional information indicating the position, type, and bonding of each atom included in the structural formula (step S602). The feature generating means 63 generates the features of each molecule based on the three-dimensional information (step S603). The physical property prediction means 64 predicts the physical properties of the molecule using the generated features (step S604).

According to the physical property prediction device 60 of the second example embodiment, it is possible to generate features based on input information indicating the structural formula of the molecule, and to predict the physical properties of the molecule using the generated features.

A part or all of the example embodiments described above may also be described as the following supplementary notes, but not limited thereto.

### Supplementary Note 1

A physical property prediction device comprising:
an input information acquisition means configured to acquire input information indicating a structural formula of a molecule;
a three-dimensional information generation means configured to generate three-dimensional information indicating a position, type, bounding of each atom included in the structural formula;
a feature generation means configured to generate features of the molecule based on the three-dimensional information; and
a physical property prediction means configured to predict physical properties of the molecule using the features generated.

### Supplementary Note 2

The physical property prediction device according to supplementary note 1, wherein
the molecule is a macromolecule, and
the physical property prediction device further comprises a division means configured to divide a structural formula of the macromolecule into a plurality of units,
wherein the three-dimensional information generation means generates the three-dimensional information of each unit.

### Supplementary Note 3

The physical property prediction device according to supplementary note 1, wherein the feature generation means acquires, as the features, molecular descriptors representing physicochemical characteristics of the structural formula by applying a predetermined rule to the three-dimensional information.

### Supplementary Note 4

The physical property prediction device according to supplementary note 3, further comprising a feature selection means configured to select specific molecular descriptors by performing a feature selection with respect to molecular descriptors corresponding to the three-dimensional information,
wherein the physical property prediction means predicts the physical properties of the molecule using the molecule descriptors selected by the feature selection means.

### Supplementary Note 5

The physical property prediction device according to supplementary note 4, wherein the feature selection means selects only molecular descriptors which are independent of each other as specific molecular descriptors by performing a correlation analysis as the feature selection.

### Supplementary Note 6

The physical property prediction device according to supplementary note 4, wherein the feature selection means selects a predetermined number of molecular descriptors from high importance using a feature selection library.

Supplementary Note 7

The physical property prediction device according to supplementary note 4, wherein the feature selection means acquires a selection result of features by a user as the feature selection, and selects specific molecular descriptors based on the selection result.

### Supplementary Note 8

The physical property prediction device according to supplementary note 4, further comprising a relevant information acquisition means configured to acquire relevant information related to the molecule,
wherein the feature selection means selects specific molecular descriptors based on the relevant information.

### Supplementary Note 9

The physical property prediction device according to supplementary note 1, wherein the physical property prediction means predicts physical properties of the molecule based on the features using a physical property prediction model trained by machine learning in advance.

### Supplementary Note 10

A physical property prediction method comprising:
acquiring input information indicating a structural formula of a molecule;
generating three-dimensional information indicating a position, type, bounding of each atom included in the structural formula;
generating features of the molecule based on the three-dimensional information; and
predicting physical properties of the molecule using the features generated.

### Supplementary Note 11

acquiring input information indicating a structural formula of a molecule;
generating three-dimensional information indicating a position, type, bounding of each atom included in the structural formula;
generating features of the molecule based on the three-dimensional information; and
predicting physical properties of the molecule using the features generated.

While the disclosure has been described with reference to the example embodiments and examples, the disclosure is not limited to the above example embodiments and examples. It will be understood by those of ordinary skill in the art that various changes in form and details may be made therein without departing from the spirit and scope of the present disclosure as defined by the claims.

### DESCRIPTION OF SYMBOLS

11, 21 Interface
12, 22 Processor
13, 23 Memory
14, 24 Recording medium
15, 25 Display unit
16, 26 Input unit
30 Feature generation device
31 Acquisition unit
32 Division unit
33 Feature generation unit
34 Feature selection unit
35 Structural information generation unit
36 Relevant information acquisition unit
40 Physical property prediction device
41 Physical property prediction model storage unit
42 Physical property prediction unit

What is claimed is:

1. A physical property prediction device comprising:
at least one memory configured to store instructions; and
at least one processor configured to execute the instructions to:
acquire input information indicating a structural formula of a molecule;
generate three-dimensional information indicating a position, type, bounding of each atom included in the structural formula;
generate features of the molecule based on the three-dimensional information; and
predict physical properties of the molecule using the features generated.

2. The physical property prediction device according to claim 1, wherein
the molecule is a macromolecule,
the processor is further configured to divide a structural formula of the macromolecule into a plurality of units, and
the processor three means generates the three-dimensional information of each unit.

3. The physical property prediction device according to claim 1, wherein processor acquires, as the features, molecular descriptors representing physicochemical characteristics of the structural formula by applying a predetermined rule to the three-dimensional information.

4. The physical property prediction device according to claim 3, wherein
the processor is further configured to select specific molecular descriptors by performing a feature selection with respect to molecular descriptors corresponding to the three-dimensional information,
the processor predicts the physical properties of the molecule using the molecule descriptors selected by the feature selection means.

5. The physical property prediction device according to claim 4, wherein the processor selects only molecular descriptors which are independent of each other as specific molecular descriptors by performing a correlation analysis as the feature selection.

6. The physical property prediction device according to claim 4, wherein the processor selects a predetermined number of molecular descriptors from high importance using a feature selection library.

7. The physical property prediction device according to claim 4, wherein the processor acquires a selection result of features by a user as the feature selection, and selects specific molecular descriptors based on the selection result.

8. The physical property prediction device according to claim 4, the processor is further configured to acquire relevant information related to the molecule,
wherein the processor selects specific molecular descriptors based on the relevant information.

9. The physical property prediction device according to claim 1, wherein the processor predicts physical properties of the molecule based on the features using a physical property prediction model trained by machine learning in advance.

10. A physical property prediction method comprising:
acquiring input information indicating a structural formula of a molecule;
generating three-dimensional information indicating a position, type, bounding of each atom included in the structural formula;
generating features of the molecule based on the three-dimensional information; and
predicting physical properties of the molecule using the features generated.

11. A non-transitory computer readable recording medium storing a program, the program causing a computer to perform a process comprising:
acquiring input information indicating a structural formula of a molecule;
generating three-dimensional information indicating a position, type, bounding of each atom included in the structural formula;
generating features of the molecule based on the three-dimensional information; and
predicting physical properties of the molecule using the features generated.
